(19) United States
(12) Patent Application Publication
(10) Pub. No.: US 2004/0265795 A1
(43) Pub. Date: Dec. 30, 2004

(54) Procedure for quantitative determination of viral or bacterical particles having a cholesterol-containing envelope

(76) Inventors: Manfred Wirth, Braunschweig (DE); Christine Beer, Braunschweig (DE)

Correspondence Address: Frommer Lawrence & Haug, 745 Fifth Avenue, New York, NY, 10151, US

(21) Appl. No.: 10/492,895

(22) PCT Filed: Oct. 17, 2002
(86) PCT No.: PCT/EP02/11640

(30) Foreign Application Priority Data
Oct. 18, 2001 (DE) 10150959.6

(51) Int. Cl. C12Q 1/70; C12Q 1/04

(52) U.S. Cl. 435/5; 435/34

(57) ABSTRACT

The invention relates to a procedure for quantitatively determining viral or bacterial particles having a cholesterol-containing envelope, wherein the viral particles under a fluorescence microscope.

The invention relates to a procedure for quantitative determination of viral or bacterial particles having a cholesterol-containing envelope, using the fluorescent dye filipin.

### BACKGROUND

Virus Quantification

Quantifying total viral particles is a relatively involved procedure compared to determining the number of infectious viral particles. Detection of retroviral particles in cell culture supernatants is used, on the one hand, to exclude the possibility of contamination with retroviruses and, on the other hand, to check the quantity and quality of virus production by packaging cells. Contamination with retroviruses is usually detected by means of the activity of viral reverse transcriptase in the supernatant [85, 106] (a literature list, including bibliographic details, will be found at the end of the description). There are various methods available for quantifying the production of total viral particles. Besides the afore-mentioned activity of reverse transcriptase, determination of viral “strong-stop” cDNA, electron microscope investigations and immunohistochemical staining of proteins on the virus surface are used for quantifying total particles [7, 84., 85, 103].

Those methods can be categorised, according to their principle, into two types. On the one hand, they consist of indirect quantification methods wherein the number of viral particles is inferred from viral enzyme activities or from the nucleic acid content. On the other hand, there are direct methods, serving to make the viral particles visible. The first category includes detection of viral RNA, of viral “strong-stop” cDNA and of the activity of viral reverse transcriptase [84, 85, 143, 144]. Electron microscope investigations and immunohistochemical staining of viral envelope proteins on the surface of the virus are included in the second category of methods used [103, 7]. The advantages of the indirect methods lie in the fact that they are relatively simple to perform and a large number of samples can be quantified rapidly. The disadvantage of that kind of particle number determination lies in the fact that it provides only an approximate idea of the number of viruses released and, as has been shown in the context of investigations relating to the invention, factors specific to the cell type are in some cases required to calculate the total particle numbers. It is, for example, usually assumed that 1% of infectious retroviruses contains “strong-stop” cDNA, irrespective of the cell line and production conditions. However, the data presented together with the invention show that the number of infectious viruses as a proportion of the cDNA-containing particles varies in dependence upon the type of production cell line. For the murine fibroblast cell line NIH3T3, it has been possible to demonstrate, in a number of experiments, that approximately half the infectious particles contain “strong-stop” cDNA. That contradicts the data of other study groups, which have shown that about 1% of total particles are infectious [84, 149]. In order to quantify total viral particles exactly using this method, therefore, it is necessary to take into account a factor specific to the cell type. That factor can, however, be determined only using methods that, like the direct methods, are capable of detecting all viral particles. However, electron microscope investigations, for example, require a not inconsiderable outlay on apparatus and materials. The immunohistochemical staining procedures developed by Pizzato et al. [7] can, in contrast, be performed relatively rapidly and simply, but they do, in some cases, require a considerable outlay in optimising the staining procedures and they are dependent upon the existence of a corresponding antibody to viral surface proteins.

Principle of Staining

When retroviruses are formed and released, the viral membrane is formed from the plasma membrane of the production cell. In terms of its composition, that viral envelope is similar to the membrane regions in which budding of the virus occurred [46], with one of the constituents, besides proteins, sphingolipids and glycolipids, being cholesterol [150]. The latter lipid, to which filipin binds specifically, is an essential constituent of eukaryotic cells. Filipin has been isolated from the culture supernatant of Streptomyces filipinensis; it consists of a 35-membered lactone ring and accordingly belongs to the group of the polyene macrolide antibiotics [145]. Earlier studies have shown that filipin forms a complex with the cholesterol of the membrane of fibroblasts [151] or fat cells [152] or the external envelope of retroviruses and other enveloped viruses [146, 147], the viral membranes being used in those studies for investigation of protein-lipid interactions with the aid of electron micrographs.

The invention aims to provide a procedure or method for quantitatively determining viral and bacterial particles that can be carried out rapidly and simply but is nevertheless reliable.

That aim is achieved in accordance with the invention by a method of quantifying viral or bacterial particles having a cholesterol-containing envelope, wherein the particles are stained with a fluorochromic or fluorogenic substance which binds to the cholesterol-containing envelope, and the fluorescence signals of the individual particles are then quantitatively determined.

In accordance with the invention, the method can be applied to retroviruses, ortho-myxoviruses, paramyxoviruses, arteri-viruses, togaviruses, bunyaviruses, rhabdoviruses, filoviruses, arenaviruses, coronaviruses, herpesviruses, flaviviruses, hapad-naviruses, poxvirues or iridoviruses.

In accordance with the invention, the method can be applied to, especially HIV, measles virus, influenza virus, murine leukaemia virus, murine leukaemia virus pseudotype or mycoplasmas.

In the method according to the invention, the number of particles is determined by counting fluorescent particles under an optical light microscope or a fluorescence microscope.

Furthermore, in the method according to the invention, a fluorochromic or fluorogenic substance selected from the following group can be used:

(a) fluorescent cholesterol binding substance,

(b) coupling product of a fluorescent cholesterol binding substrate and a fluorescent dye; or

(c) coupling product of a cholesterol binding substrate without fluorescence and a fluorescent dye.

Furthermore, in the method according to the invention a polyene macrolide or a dimethylaminonaphthaline derivative can be used as fluorescent cholesterol binding substance according to A.

Furthermore, in the method according to the invention filipin, amphotericin B, nystatin or pimaricine can be used as polyene macrolide.

Furthermore, in the method according to the invention filipin can be used as polyene macrolide.

Furthermore, in the method according to the invention the filipin fluorescence can be excited at a wavelength of 387±14 nm and the counting can be carried out at the emission wavelength of 450±29 nm.

Furthermore, in the method according to the invention a polyene macrolide can be used as a fluorescent cholesterol binding substrate according to B.

Furthermore, in the method according to the invention filipin, amphothericin B. nystatin or pimaricine can be used as polyene macrolide.

Furthermore, in the method according to the invention a saponine or a bacterial toxin can be used as cholesterol binding substance according to C.

Furthermore, in the method according to the invention digitonin, tomatin or convallamarin can be used as saponine.

Furthermore, in the method according to the invention perfringolysin-O can be used as bacterial toxin.

Furthermore, in the method according to the invention a fluorescent dye selected from the following group can be used: OH-reactive fluorescent dye; OH/NH₂-fluorescent dye; or OH/NH₂/SH-fluorescent dye.

Furthermore, in the method according to the invention fluorescein-5-carbonyl-azide or dansylchloride can be used as OH-reactive fluorescent dye.

Furthermore, in the method according to the invention a bodipy dye or a Alexa-dye can be used as OH/NH₂-reactive fluorescent dye.

Furthermore, in the method according to the invention a monobromobiman or demethylmonobromobiman can be used as OH/NH₂/SH-reactive fluorescent dye.

Furthermore, in the method according to the invention for quantitative determination, the number and/or concentration of fluorescent particles can be compared to the known number and/or known concentration of specified fluorescent particles.

Furthermore, in the method according to-the invention for comparison, fluorescent particles can be specified that are from 0.5 times to twice as large as, and especially about the same size as, the particles being quantified.

Furthermore, in the method according to the invention for comparison, inert fluorescent particles can be specified.

Finally, in the method according to the invention fluorescent particles can be specified that are provided with the same or a different fluorochromic or fluorogenic substance as the particles being quantified.

The method of quantifying viruses developed in the context of the present invention combines the advantages of antibody-independent staining of the viral or bacterial membrane and quantification with the aid of a fluorescence microscope.

The interactions between cholesterol and a fluorochromic or fluorogenic substance which binds to a cholesterol-containing envelope have not hitherto been used for quantifying viral particles by means of fluorescence using a fluorescence microscope, one of the reasons probably being that the possibility of using high-quality lenses of about 1000× magnification for making viruses or bacteria visible has been underestimated.

The invention relates further to a kit of parts for quantifying viral or bacterial particles having a cholesterol-containing envelope, which comprises

a fluorochromic or fluorogenic substance that binds to the cholesterol-containing envelope, and

(as optional constituent) fluorescent particles as reference standard.

In the kit of parts according to the invention, the reference standard can be present in an aqueous medium.

Furthermore, in the kit of parts according to the invention, the fluorescent particles of-the reference standard can be inert particles.

Furthermore, in the kit of parts according to the invention, the fluorescent particles of the reference standard can be from 0.5 times to twice as large as, and especially about the same size as, the particles being quantified.

Furthermore, the kit of parts according to the invention may comprise a fluorochromic or fluorogenic substance that binds to a cholesterol-containing virus or bacterial particle envelope as described for the method according to the invention, for quantifying viral or bacterial particles having a cholesterol-containing envelope.

Furthermore, in the kit of parts according to the invention, the fluorochromic or fluorogenic substance that binds to the cholesterol-containing envelope of particles that are specified, may be the same or a different substance the fluorescent particles are provided with.

The invention relates finally to a use of a fluorochromic or fluorogenic substance that binds to a cholesterol-containing envelope of viral or bacterial particles as described for the method according to the invention, for quantifying viral or bacterial particles having a cholesterol-containing envelope.

The invention is illustrated, without limitation, in greater detail hereinbelow, with reference to Examples and Figures.

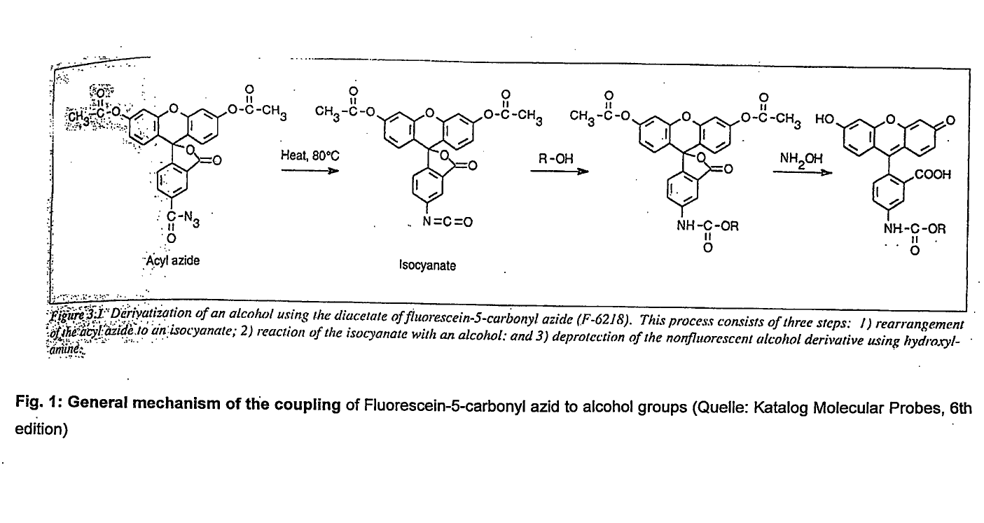

FIG. 1: General mechanism of the coupling of fluorescein-5-carbonyl azide to alcohol groups (Quelle: Katalog Moelcular Probes, 6^{th} edition) Derivatization of an alcohol using the diacetate of fluorescin-5-carbonyl azide (F-6218). This process consits of three steps: 1) rearrangement of the acyl azide to an isocynate; 2) reaction of the isocynate with an alcohol; and 3) deprotection of the nonfluorescent alcohol derivative using hydroxylamine.

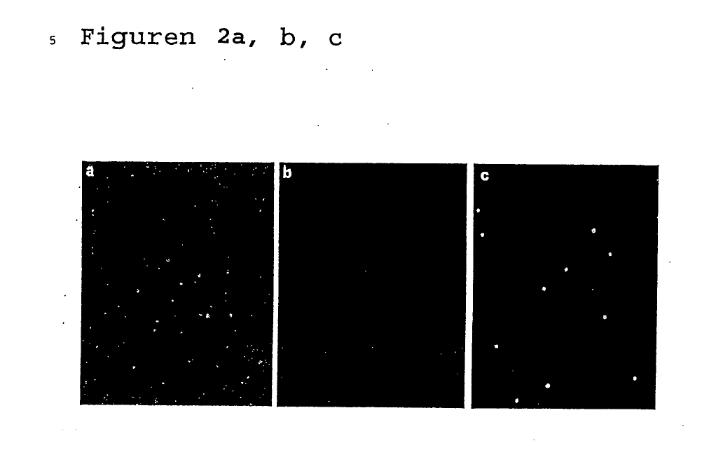

FIG. 2: Labelling of the viral membrane of 4070A MLV's with filipin. a) 4070A MLV produced by NIH3T3 after transfection with the 4070A MLV provirus and the retroviral vector pLEIN. After fixing on glass slides by means of Polybrene, the viruses were labelled with filipin; b) negative control; supernatant of mock-transfected NIH3T3; c) Texas Red-labelled beads 100 nm in size. The beads were likewise fixed on glass slides using Polybrene and serve as a reference for the size of the viral particles, which are about 100 nm in size, and as a standard for quantification; fluorescence micrographs, original magnification 1000× (oil immersion lens).

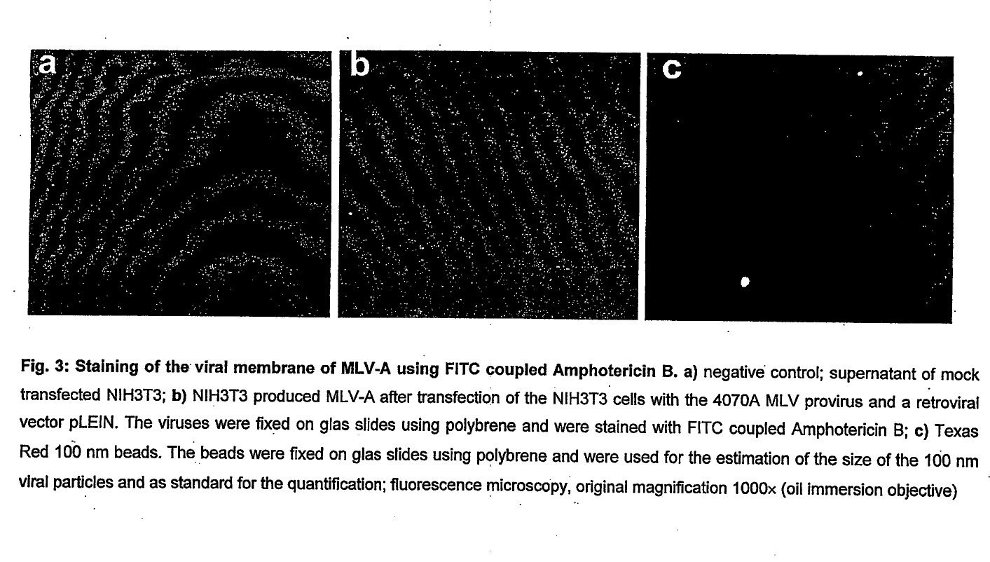

FIG. 3: Staining of the viral membrane of MLV-A using FITC coupling Amphotericin B. a) negative control; supernatant of mock transfected NIH3T3; b) NIH3T3 produced MLV-A after transfection of the NIH3T3 cells with the 4070A MLV provirus and a retroviral vector PLEIN. The viruses were fixed on glass slides using polybrene and were stained with FITC coupled Amphotericin B; c) Texas Red 100 nm beads. The beads were fixed on glass slides using polybrene and were used for the estimation of the size of the 100 nm viral particles and as standard for the quantification; fluorescence microscopy, original magnification 1000× (oil immersion objective).

### EXPERIMENTAL PART 1

Staining of Viral Particles, using the Example of 4070A MLV

The fluorogenic properties of filipin are utilised for quantifying viral particles. Viruses labelled with filipin can be made visible and quantified under a fluorescence microscope (excitation 387 nm; emission 450 nm) (see FIG. 2). Quantification of the physical viral particles is carried out by direct comparison with Texas Red-labelled beads added in a known concentration to the virus-containing supernatant before fixing (see Methodology chapter, Quantification section).

Advantages of Filipin Staining Over Other Methods of Quantification

Filipin staining has a number of advantages: it can be performed simply and rapidly (taking less than 2 hours). Comparison with the quantification of “strong-stop” CDNA shows that it is up to 40 times more sensitive (compare Table 1). Because of its specificity for cholesterol, the method does not require any lengthy optimisation as is frequently necessary in the case of immunohistochemical staining procedures, and it is not dependent on virus-specific antibodies, which is advantageous, in particular, in the case of those viruses for which antibodies are not available. The viral particles can be made visible using a conventional fluorescence microscope of a kind that can be found in any biological establishment. The sole condition is that 1000× magnification is possible and an appropriate filter set is available, both being possible, however, with a simple microscope outfit. A further advantage of this particle staining procedure is that it can be used generally for any type of virus that has a cholesterol-containing envelope (e.g. HIV, measles virus, influenza virus).

TABLE 1
Comparison of particle number determination using
cDNA measurement and filipin labelling of 4070A
MLV.

| | | filipin-labelled |
|---|---|---|
| | cDNA particles | particles |
| | [particles/10⁶ | [particles/10⁶ |
| Cell line | cells/24 h] | cells/24 h] |
| NIH3T3 | 3.1 × 10⁷ | 1.5 × 10⁸ |
| BHK-A | 3.7 × 10⁶ | 5.8 × 10⁷ |
| BHK 21B | 6.0 × 10⁶ | 2.6 × 10⁷ |
| TE FlyA7 | 8.2 × 10⁵ | 3.2 × 10⁷ |

Determination of the Variance of Quantification of Filipin-Labelled Viral Particles

In order to use filipin labelling for quantifying physical viral particles, it must be ensured that the analyses are reproducible. For that purpose, nine viral 4070A MLV samples were stained, with duplicate determinations being carried out, and each individual staining was quantified. The average deviation from the mean was calculated from the data. The average deviation was 12.5%.

Methodology

Staining of Viral Membranes

For systematically quantifying viruses enveloped by the plasma membrane of the host cell, there has been developed a staining method that is not dependent on antibodies because the cholesterol of the viral membrane is fluorescence-labelled in targeted manner. Filipin is an antibiotic which acts on the membrane sterols of eukaryotic cells, leading to changes in membrane permeability. By virtue of its fluorescence properties and specific binding to cholesterol, it can be used for staining cholesterol-containing membranes.

For staining, 195 μl of virus-containing cell supernatant (filtered, 0.45 μm) were mixed with 4 μl of Polybrene (0.4 mg/ml) and 1 μl of Texas Red-labelled fluorescence particles (Molecular Probes, diameter 100 nm, 3.6×10⁷/μl) and incubated for 1 hour at room temperature in chamber slides provided with 8 recesses. The samples were then washed three times with 1XPBS and were incubated with glycine (1.5 mg/ml) for 10 min and then with filipin (0.05 mg/ml) for 30 min. The specimens were washed three times with 1XPBS; fluorescence enclosure medium (Dako) was poured over the specimens and they were sealed with a glass coverslip.

1XPBS: 140 mM NaCl; 27 mM KCl; 7.2 mM NaHPO₄; 14.7 mM

KH2PO₄; pH 6.8-7.0 as 1033 stock solution from GibcoBRL; solution for use diluted 1:10 with distilled water

Detection of Stained Viruses

The number of viral particles in a virus-containing cell supernatant is determined with the aid of fluorescence micrographs (1000× magnification, 100× oil immersion lens). Visualisation of the fluorescent particles was carried out using filters of the wavelengths 595/615. The filipin signals can be detected with the aid of a fluorescence microscope (Filter Set XF113 from Omegafilters, excitation 387 nm (28), emission 450 nm (58)).

Quantification of Filipin-labelled Viral Particles

For quantification, micrographs are taken, from the same specimen recess, of the filipin-labelled viruses and the fluorescence-labelled beads. For a statistically significant evaluation of quantification, at least seven micrographs should be taken (both for viruses and for beads). The viruses and beads visible on the images are counted. Determination of the concentration of viruses is carried out using formula 1. Calculation is made possible by using beads in a known concentration.

$$\frac{{C}_{beads} \times {A}_{virus}}{{A}_{beads}} = {C}_{virus} \qquad ( 1 )$$

C_{beads}—concentration of beads (number/ml)

Cᵥᵢᵣᵤₛ—concentration of viruses

A_{beads}—number of beads per image

Aᵥᵢᵣᵤₛ—number of viruses per image

In summary, in accordance with the invention, a new method has been developed for quantifying viruses that have a cholesterol-containing membrane. The method consists of an antibody-independent staining procedure wherein the cholesterol molecules of the viral membrane are specifically labelled by the antibiotic filipin. Filipin has fluorogenic properties and can be visualised using a filter set for bfp (blue fluorescence protein; excitation at 387 nm, emission at 450 nm). As a result of that fluorescence, the viruses can be made visible using a fluorescence microscope at 1000× magnification. Quantification is carried out by direct comparison with fluorescence-labelled (e.g. Texas Red-labelled) particles 100 nm in size, the concentration of which is known.

### EXPERIMENTAL PART 2

The staining of viruses and bacteria having a cholesterol-containing envelope (e.g. retroviruses, Mycoplasma) can be carried out with naturally fluorescent, cholesterol binding substances or with synthetic compounds, which were modified with fluorescent dyes. As with the staining with filipin the quantification takes place using a fluorescence microscope with a 1000× magnification and standard beads. (The choice of the fluorescence filters depends on the fluorescence properties of the used fluorescent dyes (excitation/emission)). On the one hand the coupling of the fluorescent dyes to the cholesterol binding substances serves as improvement of the naturally existing fluorescence. On the other hand the coupling of the fluorescent dyes to the cholesterol binding substances enables the usage of cholesterol binding substances which naturally have no fluorescence.

A. Fluorescent Cholesterol Binding Substrates:

Polyene macrolide: Filipin, Amphotericin B, Nystatin, Pimaricin

Di-methyl-aminonaphtaline: Prodan, Laurdan

B. Fluorescent Cholesterol Binding Substances with Improved Fluorescence Properties after Coupling of Fluorescent Dyes:

Polyene macrolide: Filipin, Amphotericin B, Nystatin, Pimaricin

C. Non-fluorescent Cholesterol Binding Substances which can be used for Modification with Fluorescent Dyes:

Saponine: Digitonin, Tomatin, Convallamarin Bacterial toxins: Perfringolysin-O

D. Fluorescent Dyes are:

OH reactive fluorescent dyes like Fluorescein-5-carbonyl-azide (FITC) (EM 518 nm, Ex 492±20 nm; provider: Molecular Probes), dansylchloride (EM 520 nm, EX 350±20 nm) or OH, NH₂, SH reactive substances like monobrombiman (EM 464 nm, Ex 390 nm; provider Sigma) or De-methyl-monobrombiman (AG Dr. Hofle, GBF). Possibly OH, NH₂ reactive Bodopy or Alexa-dyes (Molecular Probes)

2. Coupling of Fluorescent Dyes to Cholesterol Binding Substances

### EXAMPLE

### Amphotericin-B-FITC

As example for the general feasibility of the coupling of fluorescent dyes to cholesterol binding substances Fluorescein-5-carbonyl-azide was coupled to the polyene macrolide Amphotericin B as shown in FIG. 1. The alcohol groups of the Amphotericin B reacts with the FITC following the mechanism in shown in FIG. 1.

0,5 mg/ml Fluorescein-5-carbonyl-azid were dissolved in dimethylformamide and after mixing with Amphotericin-B heated to 80° C. During this step the OH-groups of the Amphotericin-B were coupled to the resulting isocyanate of the Fluorescein-5-carbonyl-azide. After adding hydroxylamine the fluorescence of the Fluorescein-5-carbonyl-azide was deprotected. The coupling to Amphotericin-B was determined using thin layer chromatography.

3. Quantitative Determination of MLV-A using Amphotericin-B-FITC and Fluorescence Microscopy

The FITC coupled Amphotericin-B was used for the staining of amphotropic mouse leukemia viruses (MLV-A) produced from the mouse fibroblast cell line NIH3T3. 195 μl virus containing cell supernatant (filtrated: 0.45 μm), 4 μl polybrene (0.4 mg/ml) and 1 μl Texas Red fluorescent beads as reference standard (Molecular Probes, 100 nm, 3.6×10⁷/μl) were mixed for the staining and incubated on 8 well Chamber-slides for 1 hour at room temperature. The probes were washed three times with 1 ×PBS, incubated with glycine (1.5 mg/ml) for 10 min and afterwards with FITC coupled Amphotericin B (4 μg) incubated for 1 hour. The probes were washed three times with 1×PBS, overlaid with fluorescence counting medium (Dako) and a glass slide.

FIG. 3 shows a typical staining. Therefore, it was possible to use the novel FITC coupled Amphotericin-B for the detection and quantification of viral particles.

Definitions, Abbreviations

4070A MLV: murine leukaemia virus which is capable of infecting the cells of a large number of organisms reverse transcriptase: enzyme for the transcription of RNA into DNA

“strong-stop” CDNA: formed by reverse transcriptase in viral particles of 4070A MLV and used for quantifying viral particles

immunohistochemical staining procedures: fluorescence-labelled antibodies bind to specific surface structures and can be made visible under a fluorescence microscope

beads: term for spherical particles

Literature,

[7] Pizzato M. et al., Initial binding of murine leukemia virus particles to cells does not require specific Env-receptor interaction, J. Virol. 73: 8599-8611 (1999)

[46] Hammerstedt M. et al., Minimal exclusion of plasma membrane proteins during retroviral envelope formation, Proc. Natl. Acad. Sci. USA 97: 7527-7532 (2000)

[84] Towers G. J. et al., One step screening of retroviral producer clones by real time quantitative PCR, J. Gene Med. 1: 352-359 (1999)

[85] Muller K. et al., Real time detection of retroviruses by PCR, J. Gene Med. Supplement to volume 1: 46 (1999)

[103] Bierley S. T. et al., A comparison of methods for the estimation of retroviral burden, Dev. Biol. Stand. 88: 163-165 (1996)

[106] Goff S. et al., Isolation and properties of Moloney murine leukemia virus mutants: use of a rapid assay for release of virion reverse transcriptase, J. Virol. 38: 239-248 (1981)

[143] Hofmann-Lehmann R. et al., Sensitive and robust one-tube real-time reverse transcriptase-polymerase chain reaction to quantify SIV RNA load: comparison of one- versus two-enzyme systems, AIDS Res. Hum. Retroviruses 16: 1247-1257 (2000)

[144] Schutten M. et al., Development of a real-time quantitative RT-PCR for the detection of HIV-2 RNA in plasma, J., Virol. Methods 88: 81-87 (2000)

[145] Whitefield G. B. et al., J. Am. Chem. Soc. 77: 4799-4801 (1955)

[146] Majuk Z. et al., Effects of filipin on the structure and biological activity of enveloped viruses, J. Virol. 24: 883-892 (1977)

[147] Feltkamp C. A. et al., Membrane-associated proteins affect the formation of filipin-cholesterol complexes in viral membranes, Exp. Cell. Res. 140: 289-297 (1982)

[149] Andersen K. B. et al., Entry of murine retrovirus into mouse fibroblasts, Virology 125: 85-98 (1983)

[150] Aloia R. C. et al., Lipid composition and fluidity of the human immuno deficiency virus envelope and host cell plasma membranes, PNAS USA 90: 5181-5185 (1993)

[151] Ledvinova, J. & Elleder, M., Filipin test for diagnosis of Niemann-Pick disease type C, Sbornik Lekarsky 94 (2) 127-43 (1993)

[152] Prattes, S. et al., Intracellular distribution and mobilization of unesterfied cholesterol in adipocytes:

Triglyceride droplets are surrounded by cholesterol-rich ER-like surface layer structures. Journal of Cell Science, Vol. 113, No. 17: 2977-2989 (2000)

1. Method of quantifying viral or bacterial particles having a cholesterol-containing envelope, wherein the particles are stained with a fluorochromic or fluorogenic substance which binds to the cholesterol-containing envelope, and the fluorescence signals of the individual particles are then quantitatively determined.

2. Method according to claim 1, wherein the method is applied to retroviruses, ortho-myxoviruses, paramyxoviruses, arteri-viruses, togaviruses, bunyaviruses, rhabdoviruses, filoviruses, arenaviruses, coronaviruses, herpesviruses, flaviviruses, hapadnaviruses, poxvirues or iridoviruses.

3. Method according to claim 1, wherein the method is applied to HIV, measles virus, influenza virus, murine leukaemia virus, murine leukaemia virus pseudotype or mycoplasmas.

4. Method according to claim 1, wherein the number of particles is determined by counting fluorescent particles under an optical-light microscope or a fluorescence microscope.

5. Method according to claim 1, wherein a fluorochromic or fluorogenic substance selected from the following group is used:
(a) fluorescent cholesterol binding substance,
(b) coupling product of a fluorescent cholesterol binding substrate and a fluorescent dye; or
(c) coupling product of a cholesterol binding substrate without fluorescence and a fluorescent dye.

6. Method according to claim 5, wherein a polyene macrolide or a dimethylaminonaphthaline derivative is used as fluorescent cholesterol binding substance according to A.

7. Method according to claim 6, wherein filipin, amphotericin B, nystatin or pimaricine is used as polyene macrolide.

8. Method according to claim 7, wherein filipin is used as polyene macrolide.

9. Method according to claim 8, wherein the filipin fluorescence is excited at a wavelength of 387±14 nm and the counting is carried out at the emission wavelength of 450±29 nm.

10. Method according to claim 5, wherein a polyene macrolide is used as a fluorescent cholesterol binding substrate according to B.

11. Method according to claim 10, wherein filipin, amphothericin B, nystatin or pimaricine is used as polyene macrolide.

12. Method according to claim 5, wherein a saponine or a bacterial toxin is used as cholesterol binding substance according to C.

13. Method according to claim 12, wherein digitonin, tomatin or convallamarin is used as saponine.

14. Method according to claim 12, wherein perfringolysin-O is used as bacterial toxin.

15. Method according to claim 5, wherein a fluorescent dye selected from the following group is used: OH-reactive fluorescent dye; OH/NH₂-fluorescent dye; or OH/NH₂/SH-fluorescent dye.

16. Method according to claim 15, wherein fluorescein-5-carbonyl-azide or dansylchloride is used as OH-reactive fluorescent dye.

17. Method according to claim 15, wherein a bodipy dye or a Alexa-dye is used as OH NH₂-reactive fluorescent dye.

18. Method according to claim 15, wherein a monobromobiman or demethylmonobromobiman is used as OH/NH₂/SH-reactive fluorescent dye.

19. Method according to claim 1, wherein for quantitative determination, the number and/or concentration of fluorescent particles is compared to the known number and/or known concentration of specified fluorescent particles.

20. Method according to claim 19, wherein for comparison, fluorescent particles are specified that are from 0.5 times to twice as large as, and especially about the same size as, the particles being quantified.

21. Method according to claim 19, wherein for comparison, inert fluorescent particles are specified.

22. Method according to claim 19, wherein fluorescent particles are specified that are provided with the same or a different fluorochromic or fluorogenic substance as the particles being quantified.

23. Kit of parts for quantifying viral or bacterial particles having a cholesterol-containing envelope, which comprises
a fluorochromic or fluorogenic substance that binds to the cholesterol-containing envelope, and
(as optional constituent) fluorescent particles as reference standard.

24. Kit of parts according to claim 23, wherein the reference standard is present in an aqueous medium.

25. Kit of parts according to claim 23, wherein the fluorescent particles of the reference standard are inert particles.

26. Kit of parts according to claim 23, wherein the fluorescent particles of the reference standard are from 0.5 times to twice as large as, and especially about the same size as, the particles being quantified.

27. Kit of parts according to claim 23, comprising a fluorochromic or fluorogenic substance that binds to a cholesterol-containing virus or bacterial particle envelope, for quantifying viral or bacterial particles having a cholesterol-containing envelope.

28. Kit of parts according to claim 23, wherein the fluorochromic or fluorogenic substance that binds to the cholesterol-containing envelope of particles that are specified, is the same or a different substance the fluorescent particles are provided with.

29. Use of a fluorochromic or fluorogenic substance that binds to a cholesterol-containing envelope of viral or bacterial particles, according to claim 1, for quantifying viral or bacterial particles having a cholesterol-containing envelope.
